(19) United States
(12) Patent Application Publication
(10) Pub. No.: US 2024/0383152 A1
(43) Pub. Date: Nov. 21, 2024

(54) MULTI-AXIS FORCE TRANSDUCER FEEDBACK FROM ROBOTIC END EFFECTOR ADAPTER

(71) Applicant: Medtronic Navigation, Inc., Lafayette, CO (US)

(72) Inventor: Richard Todd Hage, Castle Rock, CO (US)

(21) Appl. No.: 18/656,673

(22) Filed: May 7, 2024

Related U.S. Application Data
(60) Provisional application No. 63/466,629, filed on May 15, 2023.

(51) Int. Cl. B25J 13/08; A61B 34/30; B25J 9/16

(52) U.S. Cl. CPC B25J 13/085; A61B 34/30; B25J 9/1674; B25J 13/088

(57) ABSTRACT

A system according to at least one embodiment of the present disclosure includes a robotic arm that includes a distal end interface; a force transducer couplable to the distal end interface and that measures forces exerted on the force transducer in up to six degrees of freedom; and an end effector configured to be in force-transmitting contact with the force transducer.

### CROSS-REFERENCE TO RELATED APPLICATION

This application claims the benefit of and priority to U.S. Provisional Patent Application No. 63/466,629 filed May 15, 2023, the entire disclosure of which is incorporated by reference herein.

### BACKGROUND

The present disclosure is generally directed to surgical procedures, and relates more particularly to monitoring surgical component deflection during surgical procedures.

Surgical robots may assist a surgeon or other medical provider in carrying out a surgical procedure, or may complete one or more surgical procedures autonomously. Providing controllable linked articulating members allows a surgical robot to reach areas of a patient anatomy during various medical procedures.

### BRIEF SUMMARY

Example aspects of the present disclosure include:

A system according to at least one embodiment of the present disclosure comprises: a robotic arm that includes a distal end interface; a force transducer couplable to the distal end interface and that measures forces exerted on the force transducer in up to six degrees of freedom; and an end effector configured to be in force-transmitting contact with the force transducer.

Any of the features herein, wherein the end effector is connectable to a surgical tool.

Any of the features herein, further comprising: a processor; and a memory coupled to the processor and storing data thereon that, when executed by the processor, enable the processor to: determine an orientation of the end effector relative to sensing axes of the force transducer; and determine an orientation of a surgical tool relative to the sensing axes of the force transducer.

Any of the features herein, wherein the data further enable the processor to: determine, when the force transducer measures a first force in a first direction and based on the orientation of the surgical tool relative to the sensing axes, a direction and an amplitude of a resulting force on the surgical tool.

Any of the features herein, wherein the data further enable the processor to: compare the resulting force to a first threshold value when the direction of the resulting force on the surgical tool is in a second direction; compare the resulting force to a second threshold value when the direction of the resulting force on the surgical tool is in a third direction; and generate, when the resulting force exceeds at least one of the first threshold value and the second threshold value, an alert indicating the surgical tool has experienced deflection.

Any of the features herein, wherein the data further enable the processor to: automatically disable, when the alert is generated, the surgical tool.

Any of the features herein, wherein the data further enable the processor to: determine, when the force transducer measures a first force in a first direction and based on the orientation of the end effector relative to the sensing axes, a direction and an amplitude of a resulting force on the end effector.

Any of the features herein, wherein the data further enable the processor to: compare the resulting force to a first threshold value when the direction of the resulting force on the end effector is in a second direction; compare the resulting force to a second threshold value when the direction of the resulting force on the end effector is in a third direction; and generate, when the resulting force exceeds at least one of the first threshold value and the second threshold value, an alert indicating the end effector has experienced deflection.

An apparatus according to at least one embodiment of the present disclosure comprises: a robotic arm with a first end; a force transducer with a first end in force-transmitting contact with the first end of the robotic arm, the force transducer measuring forces exerted on the force transducer in up to six degrees of freedom; and an end effector with a first end in force-transmitting contact with a second end of the force transducer, wherein the end effector includes a second end that is couplable to a surgical instrument.

Any of the features herein, further comprising: a processor; and a memory coupled to the processor and storing data thereon that, when executed by the processor, enable the processor to: determine an orientation of the end effector relative to sensing axes of the force transducer; and determine an orientation of a surgical instrument relative to the sensing axes of the force transducer.

Any of the features herein, wherein the data further enable the processor to: determine, when the force transducer measures a first force in a first direction and based on the orientation of the surgical instrument relative to the sensing axes, a direction and an amplitude of a resulting force on the surgical instrument.

Any of the features herein, wherein the data further enable the processor to: compare the resulting force to a first threshold value when the direction of the resulting force on the surgical instrument is in a second direction; compare the resulting force to a second threshold value when the direction of the resulting force on the surgical instrument is in a third direction; and generate, when the resulting force exceeds at least one of the first threshold value and the second threshold value, an alert indicating the surgical instrument has experienced deflection.

Any of the features herein, wherein the data further enable the processor to: automatically disable, when the alert is generated, the surgical instrument.

Any of the features herein, wherein the data further enable the processor to: determine, when the force transducer measures a first force in a first direction and based on the orientation of the end effector relative to the sensing axes, a direction and an amplitude of a resulting force on the end effector.

Any of the features herein, wherein the data further enable the processor to: compare the resulting force to a first threshold value when the direction of the resulting force on the end effector is in a second direction; compare the resulting force to a second threshold value when the direction of the resulting force on the end effector is in a third direction; and generate, when the resulting force exceeds at least one of the first threshold value and the second threshold value, an alert indicating the end effector has experienced deflection.

A surgical system according to at least one embodiment of the present disclosure comprises: a robotic arm that includes a distal end interface; a force transducer couplable to the distal end interface and that measures forces exerted on the force transducer in up to six degrees of freedom; an end effector configured to be in force-transmitting contact with the force transducer and couplable to a surgical tool; a processor; and memory coupled to the processor and storing data thereon that enable the processor to: determine an orientation of the surgical tool relative to sensing axes of the force transducer; and determine an orientation of the end effector relative to the sensing axes of the force transducer.

Any of the features herein, wherein the data further enable the processor to: determine, when the force transducer measures a first force in a first direction and based on at least one of the orientation of the surgical tool relative to the sensing axes and the orientation of the end effector relative to the sensing axes, a direction and an amplitude of a resulting force on at least one of the surgical tool and the end effector.

Any of the features herein, wherein the data further enable the processor to: compare the resulting force to a first threshold value when the direction of the resulting force is in a second direction; compare the resulting force to a second threshold value when the direction of the resulting force is in a third direction; and generate, when the resulting force exceeds at least one of the first threshold value and the second threshold value, an alert indicating at least one of the surgical tool and the end effector has experienced deflection.

Any of the features herein, wherein the data further enable the processor to: automatically disable, when the alert is generated, the surgical tool.

Any of the features herein, wherein the second direction is along an axial direction of the surgical tool, and wherein the third direction is lateral to the surgical tool.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a diagram of a robotic surgical system according to at least one embodiment of the present disclosure;

FIG. 3A is a schematic of a robotic arm with a surgical tool interacting with an anatomical element in a first configuration according to at least one embodiment of the present disclosure;

FIG. 3B is a schematic of the robotic arm with the surgical tool interacting with the anatomical element in a second configuration according to at least one embodiment of the present disclosure; and

FIG. 4 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Implementations of a robotic arm for surgery may have an adapter interface between the end effector base and the arm end linkage. By placing a multi-axis force transducer at the end effector to arm interface, real time 6 degree of freedom forces can be captured. The measured forces can then be analyzed during the procedure to alert the surgeon to excessive multi-axis forces with respect to the patient and robotic arm. Alert to excessive arm forces may allow awareness of potential excessive arm deflection. Awareness of excessive patient-imposed forces may allow awareness of potential patient spine deflections. In some embodiments, the measured forces may be analyzed post-procedure to develop awareness of typical forces imposed during procedures, which may be beneficial in designing the robotic arm. In some embodiments, a single axis force transducer may be used.

In some embodiments, an amount of arm deflection may be estimated using weight deflection and structural mechanics deflections. The estimation may use either a distributed weight load or a point load to estimate the amount of arm deflection. Due to the greater inertial moment of the robotic arm along the arm as opposed to lateral forces (e.g., forces perpendicular to the arm), forces in the direction of the arm (e.g., an axial instrument force) may need to be, for example, twice as large as forces in the lateral direction (e.g., a lateral instrument force) to result in the same amount of deflection (e.g., a deflection of the robotic arm of 2 millimeters).

In some embodiments, the a single axis end effector force transducer may measure forces along the transducer actuator axis. When the transducer axis has a known orientation relative to the instrument, a force on the instrument along the direction of the actuator axis may be captured. The single axis end effector force transducer may be used when the instrument is rigid (e.g., a drill). In other embodiments, a multi-axis end effector force transducer may be used to measure six degree of freedom forces. The forces may provide useful information regardless of the orientation of the instrument relative to the transducer axis. The multi-axis end effector force transducer may be used with a rigid drill (e.g., a drill) and/or with other instruments coupled to the end effector interface, since the multi-axis end effector force transducer captures applied moments and transverse forces.

Embodiments of the present disclosure provide technical solutions to one or more of the problems of (1) undetected forces resulting in deflections of surgical arms, surgical tools, or other surgical components and (2) undetected forces resulting in deflections of patient anatomy.

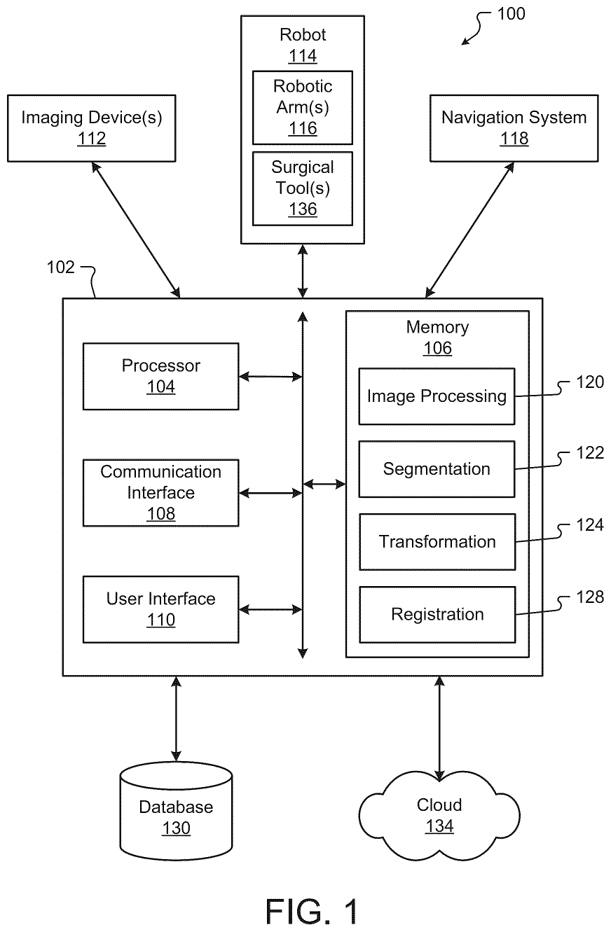

Turning first to FIG. 1, a block diagram of a system 100 according to at least one embodiment of the present disclosure is shown. The system 100 may be used to detect deflections in surgical tools or other surgical components; to control, pose, and/or otherwise manipulate a surgical mount system, a surgical arm, and/or surgical tools attached thereto; and/or carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100. For example, the system 100 may not include one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the method 400 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, segmentation 122, transformation 124, and/or registration 128. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the computing device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm 116 (as well as any object or element held by or secured to the robotic arm 116).

In some embodiments, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 comprises a surgical instrument or tool 136. The surgical tool 136 may be configured to drill, cut, saw, mill, ream, burr, etc. portions of one or more anatomical elements. For example, the surgical tool 136 may comprise a surgical drill configured to drill into the pedicles of one or more vertebra of a patient, such as during a surgical procedure that includes inserting pedicles screws into the vertebrae. In some embodiments, the surgical tool 136 may be configured to be attached to the robotic arm 116 and to be tracked or navigated by the navigation system 118. In such embodiments, the robotic arm 116 and/or the surgical tool 136 may comprise navigation markers capable of being identified and tracked by the navigation system 118. The navigation system 118 may navigate the surgical tool 136 relative to patient anatomy, enabling the surgery or surgical procedure to be performed autonomously.

The system 100 or similar systems may be used, for example, to carry out one or more aspects of the method 400 described herein. The system 100 or similar systems may also be used for other purposes.

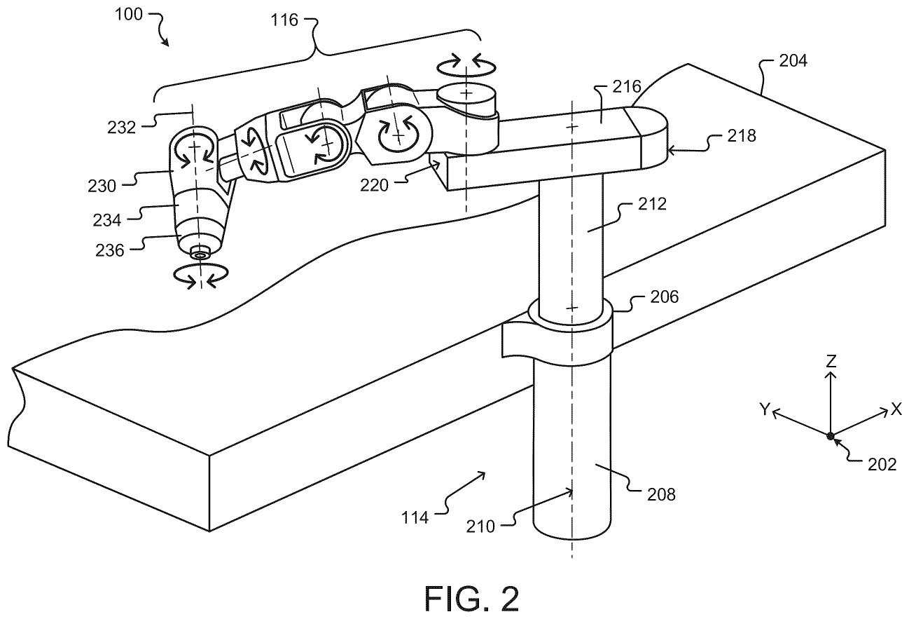

With reference to FIG. 2, a diagram of the robotic surgical system 100 and the components thereof are shown according to at least one embodiment of the present disclosure. In some embodiments, the robot 114 and the robotic arm 116 may be mounted to a surgical bed 204.

Features of the robot 114 and/or system 100 may be described in conjunction with a coordinate system 202. The coordinate system 202, as shown in FIG. 2, includes three-dimensions comprising an X-axis, a Y-axis, and a Z-axis. Additionally or alternatively, the coordinate system 202 may be used to define planes (e.g., the XY-plane, the XZ-plane, and the YZ-plane) of the robot 114 and/or robotic arm 116. These planes may be disposed orthogonal, or at 90 degrees, to one another. While the origin of the coordinate system 202 may be placed at any point on or near the components of the robot 114, for the purposes of description, the axes of the coordinate system 202 are always disposed along the same directions from figure to figure, whether the coordinate system 202 is shown or not. In some examples, reference may be made to dimensions, angles, directions, relative positions, and/or movements associated with one or more components of the robot 114 and/or robotic arm 116 with respect to the coordinate system 202.

The robot 114 and/or the robotic arm 116 may be attached to the surgical bed 204 with an attachment mechanism 206. The attachment mechanism 206 may connect the robot 114 to a first side of the surgical bed 204 such that the robotic arm 116 can access a working volume and/or one or more anatomical elements during the course of a surgery or surgical procedure. The robot 114 may comprise a base 208 that contacts a floor or other support structure in the surgical environment to provide additional support to the robot 114. In some embodiments, the base 208 may be rotatable about an axis of rotation 210 that runs along the height direction of the communication interface 108 (e.g., in the Z-axis direction of the coordinate system 202). The robot 114 may comprise a height adjustment device 212 that may be mechanically coupled with the attachment mechanism 206, the base 208, and/or the robotic arm 116. The height adjustment device 212 may be configured to move to adjust the height (e.g., the position in the Z-axis direction) of the robotic arm 116 (and/or components thereof). For instance, the height adjustment device 212 may retract into a hollow section of the base 208, such that the robotic arm 116 moves in the negative Z-axis direction (e.g., closer to the surgical bed 204 in the height direction). Similarly, the height adjustment device 212 may extend from the hollow section of the base 208 such that the robotic arm 116 moves in the Z-axis direction (e.g., further from the surgical bed 204 in the height direction).

The height adjustment device 212 may connect to a shoulder 216 of the robotic arm 116. The shoulder 216 may extend from a first end 218 to a second end 220. In some embodiments, the first end 218 may be a proximal end (e.g., an end further from the patient and closer to the surgeon) and the second end 220 may be a distal end (e.g., an end closer to the patient and further from the surgeon). The shoulder 216 may support the robotic arm 116 and/or one or more components thereof during the course of a surgery or surgical procedure. In some embodiments, the shoulder 216 may comprise one or more components of the system 100 (e.g., the navigation system 118). The shoulder 216 may be equipped with one or more motors (not shown), which may be activated or deactivated by the system 100 or components thereof (e.g., the computing device 102, the processor 104, etc.) to cause the robotic arm 116 to rotate about the axis of rotation 210. The shoulder 216 may comprise one or more cantilevers that enable the robotic arm 116 to attach to the shoulder 216. The robotic arm 116 may comprise one or more joints (e.g., pin joints, prismatic joints, ball joints, knuckle joints, cotter joints, etc.) that connect one or more cantilevers of the robotic arm 116 together and enable the robotic arm 116 to move in one, two, three, four, five, six, seven, or more degrees of freedom.

The distal end of the robotic arm 116 comprises an arm end 230, a force transducer 234, and an end effector 236. The arm end 230 may be the last or most distal cantilever of the robotic arm 116, with a proximal end of the arm end 230 connected by one or more joints to another more proximal cantilever of the robotic arm 116. The arm end 230 may include a distal end interface with an adapter onto which the force transducer 234 and/or the end effector 236 may connect. In some embodiments, the adapter interface may enable the arm end 230 to mechanically couple with the force transducer 234, such that forces on the force transducer 234 may be transferred to the arm end 230 (or vice versa).

The end effector 236 may be configured to connect or attach the surgical tool 136 to the robotic arm 116. The end effector 236 may comprise one or more interfaces on a distal end thereof that mechanically couple to the surgical tool 136, such that the end effector 236 grips or holds the surgical tool 136. The end effector 236 may also comprise a base on the proximal end thereof, with such base being couplable to the force transducer 234. In other words, the base of the end effector 236 may comprise an adapter interface the enables the end effector 236 to be placed in force-transmitting contact with the force transducer 234, such that forces experienced by the arm end 230 are transmitted to the force transducer 234.

In some embodiments, the arm end 230 may be capable of rotating, translating, or otherwise moving with respect to an end effector axis 232. The end effector axis 232 may extend through the arm end 230 in an axial direction, such that the end effector axis 232 can rotate without translating. Additionally or alternatively, the end effector axis 232 may pass through other components of the robotic arm 116, such as through the force transducer 234 and/or the arm end 230.

The force transducer 234 may be or comprise one or more force sensors configured to convert input mechanical force into an electric output signal. The force transducer 234 may, for example, detect and/or measure forces exerted thereon and provide output signals to one or more other components of the system 100, such as to the processor 104. The processor 104 may process the signal to determine the amount of force measured by the force transducer 234. Examples of the input mechanical force may include an input weight, an input tension, an input pressure, an input compression, an input load, or the like. In some embodiments, the force transducer 234 may use strain gauges to convert the input mechanical force into the electrical output signal.

The force transducer 234 may be or comprise a single axis force transducer or a multi-axis force transducer. The single axis force transducer may comprise a single sensing axis, such that the single axis force transducer detects and measures forces in the direction in which the sensing axis points or is aligned. The multi-axis force transducer, in contrast, may comprise two or more sensing axes to detect and measure forces in more than one direction. In one embodiment, the multi-axis force transducer may comprise three sensing axes, such that the multi-axis force transducer can measure forces in up to six degrees of freedom. For example, the three sensing axes can be similar to the X-axis, Y-axis, and Z-axis of the coordinate system 202, and the multi-axis force transducer measures forces in the X-axis, Y-axis, and Z-axis directions as well as torques or moments around each of the X-axis, Y-axis, and Z-axis. As previously discussed, the force transducer 234 may comprise two ends: a first end that is couplable with the distal end of the arm end 230 and a second end that is couplable with the proximal end of the end effector 236. In some embodiments, the force transducer 234 may be coupled with the arm end 230 and/or the end effector 236 such that forces experienced by the surgical tool 136, the arm end 230, and/or the end effector 236 may be detected and measured by the force transducer 234. In other words, the force transducer 234 may be disposed in force-transmitting contact with the arm end 230 and/or the end effector 236.

In some embodiments, the orientation of the arm end 230, the end effector 236, and/or the surgical tool 136 attached to the end effector 236 may be changeable relative to the sensing axes of the force transducer 234. For example, the end effector 236 may have multiple degrees of freedom to enable movement of the surgical tool 136 connected thereto, such that the orientation of the surgical tool 136 can change relative to the end effector 236. In such examples, the portion of the end effector 236 connected to and/or in force-transmitting contact with the force transducer 234 may remain fixed relative to the force transducer 234 while the surgical tool 136 can change orientation relative to the force transducer 234. As another example, the arm end 230 may be movable relative to the end effector axis 232 without moving relative to the force transducer 234. In this example, the base of the end effector axis 232 may remain fixed relative to the force transducer 234 while a more distal portion of the end effector 236 may move relative to the end effector axis 232, such as when the end effector 236 is maneuvering the surgical tool 136.

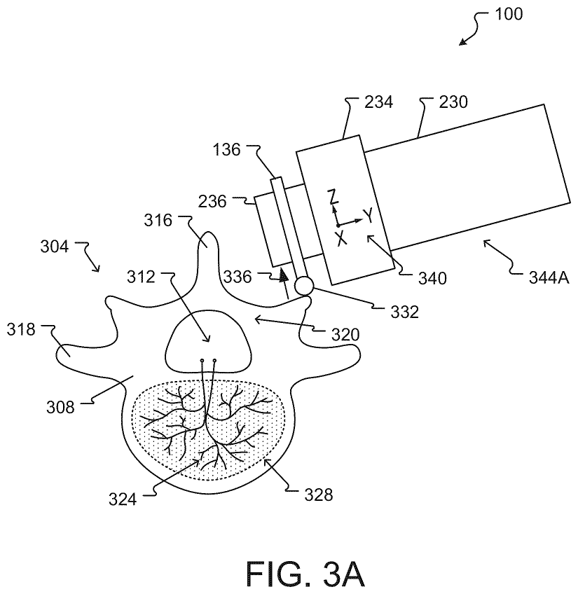

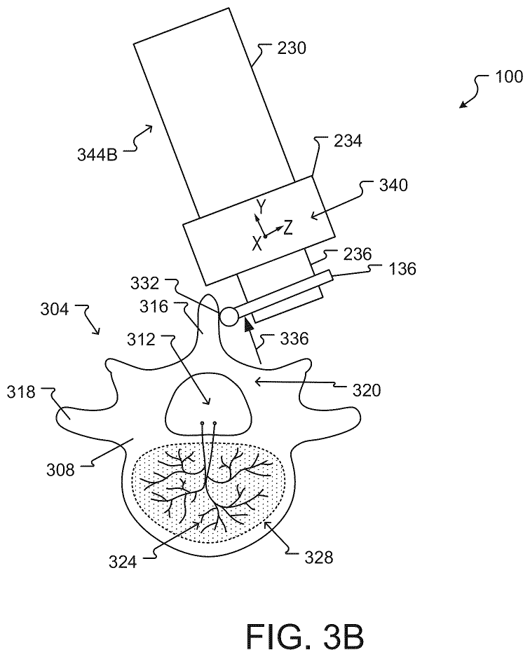

Referring now to FIGS. 3A-3B, schematics of a robotic arm with a surgical tool interacting with an anatomical element in various configurations are shown in accordance with at least one embodiment of the present disclosure. The robotic arm 116 may move the surgical tool 136 such that the surgical tool 136 interacts with a vertebra 304. As previously discussed, the surgical tool 136 can be attached to the end effector 236 and navigated relative to the vertebra 304 (e.g., using the navigation system 118). It is to be understood that, while the robotic arm 116 is depicted as interacting with a vertebra, the aspects discussed herein apply to any other anatomical element with which the robotic arm 116 interacts. The vertebra 304 comprises at least one pedicle 308, a vertebral foramen 312, a spinous process 316, a transverse process 318, a lamina 320, nerves 324, and a vertebral body area 328.

The surgical tool 136 comprises an operative portion 332. The operative portion 332 be or comprise the portion of the surgical tool 136 that manipulates, resects, and/or otherwise interacts with the vertebra 304. For example, when the surgical tool 136 comprises a surgical drill, the operative portion 332 may comprise a drill bit that drills into the pedicle 308 of the vertebra 304. In some embodiments, the navigation system 118 may navigate the operative portion 332 based on the predetermined position of the operative portion 332 relative to the surgical tool 136 and the position of the surgical tool 136 relative to the end effector 236.

One or more forces 336 may be generated as a result of the operative portion 332 interacting with the vertebra 304. The one or more forces 336 may be generated between the patient, the surgical bed 204, and/or the vertebra 304 and the operative portion 332. More generally, the force 336 may be or comprise any force generated as a result of the surgical tool 136 interacting with the patient and/or any force resulting from the operation of the robot 114 and/or the robotic arm 116 during the course of a surgery or surgical procedure.

Due to the positioning of the robotic arm 116 and/or the surgical tool 136, the one or more forces may result in deflections of the robotic arm 116 or components thereof (e.g., the arm end 230, the end effector 236, etc.) and/or the surgical tool 136. For example, the operative portion 332 may drill into the vertebra 304, and the vertebra 304 may generate a force that pushes on the surface of the operative portion 332 along an axial direction of the operative portion 332 (e.g., a force along a direction parallel to the direction of the trajectory of the drill bit into the vertebra 304). Such a force 336 may occur when the operative portion 332 is skiving on the surface of the pedicle 308 of the vertebra 304, such that the operative portion 332 is pushing against the surface of the pedicle 308 instead of drilling into the pedicle 308. Additionally or alternatively, the drilling may generate lateral forces (e.g., forces along the direction perpendicular to the direction of the drilling trajectory of the drill bit into the vertebra 304). The forces may result in unwanted deflections of the surgical tool 136. For example, the lateral force(s) may move the drill bit off the drilling trajectory, resulting in the surgical drilling at an incorrect trajectory, while the axial force(s) may make it more difficult for the drill bit to enter into and/or continue along the drilling trajectory. Additionally or alternatively, such axial and lateral forces may be transmitted to and/or experienced by the robotic arm 116. In other words, the robotic arm 116 may experience axial forces (e.g., forces along a length of the robotic arm 116) and/or lateral forces (e.g., forces perpendicular to the length of the robotic arm 116). Such forces may cause unwanted deflections in the robotic arm 116. For example, the forces may deflect the robotic arm 116 such that the robotic arm 116 and/or the surgical tool 136 is no longer correctly positioned relative to the patient and/or the vertebra 304. In some embodiments, the forces 336 may indicate that patient anatomy has moved. For example, a force 336 along the axial direction of the surgical tool 136 may indicate that the operative portion 332 is pushing against the vertebra 304. The pushing of the vertebra 304 may reposition the vertebra 304 relative to other vertebrae and/or relative to the robotic arm 116.

Different forces on the end effector 236 and/or the surgical tool 136 may be measured differently depending on the orientation of the end effector 236 and/or the surgical tool 136 relative to the force transducer 234. In other words, a force 336 in a first direction may be measured by different sensing axes 340 of the force transducer 234 depending on the orientation of the end effector 236 and/or the surgical tool 136 relative to the force 336. The sensing axes 340 may be any one or more sensing axes described herein. When the robotic arm 116 is in a first orientation 344A, the force 336 may be measured along a Z-axis direction of the sensing axes 340 of the force transducer 234, and may thus appear as a lateral force on the robotic arm 116 and an axial force on the surgical tool 136. However, when the robotic arm 116 is in a second orientation 344B different from the first orientation 344A, the same force 336 may be measured along a Y-axis direction of the sensing axes 340 of the force transducer 234, and may thus appear as an axial force on the robotic arm 116 and a lateral force on the surgical tool 136.

To account for the difference in orientation, the computing device 102 may determine the orientation of the end effector 236 and/or the surgical tool 136 relative to the force transducer 234. For example, the end effector 236 and/or the surgical tool 136 may comprise one or more pose sensors (e.g., gyroscopes and/or accelerometers) the provide information about the pose of the end effector 236 and/or the surgical tool 136 to the computing device 102. Additionally or alternatively, the navigation system 118 may track the position of the end effector 236 and/or the surgical tool 136 and provide such information to the computing device 102. Once the computing device 102 determines the orientation of the end effector 236 and/or the surgical tool 136 relative to the force transducer 234, the computing device 102 may use the force measured by the force transducer 234 to determine a resulting force applied to the arm end 230, the end effector 236, and/or the surgical tool 136. In some embodiments, the computing device 102 may determine the amplitude and/or the direction of the resulting force on the end effector 236 and/or the surgical tool 136.

The force transducer 234 may operate in a data gathering mode and/or a control mode. While in the data gathering mode, forces detected and measured by the force transducer 234 may be saved (e.g., stored in the database 130) and used to, for example, train one or more data models. The data model may be one or more artificial intelligence and/or machine learning models that receive measured forces from the force transducer 234 and other input information (e.g., orientations of the surgical tool 136 and/or the end effector 236 relative to the force transducer 234) and output a likelihood that the surgical tool 136, the end effector 236, the arm end 230, and/or patient anatomy is experiencing a deflection. In some embodiments, the data model may classify the force as either sufficient to generate a deflection or insufficient to generate the deflection. The sufficiency may be based on the information the data model receives as an input, such as operating parameters of the surgical tool 136 (e.g., the RPM of the operative portion 332), mechanical information about the robotic arm 116 (e.g., an inertial moment of the arm end 230), combinations thereof, and the like. In some embodiments, the data model may determine the force is sufficient to generate a deflection when the force exceeds a threshold value, with the threshold value representing a deflection tolerance of the system 100. When the force exceeds the threshold, the force may fall outside the operating tolerance of the system 100, and may be deemed as “excessive.” In other words, the threshold value may represent a force above which the the robotic arm 116, the surgical tool 136, or the like is treated by the system 100 as having been deflected. In some embodiments, the data models may be trained on historical data of forces measured in similar surgeries or surgical procedures.

While operating in the control mode, forces detected and measured by the force transducer 234 may be used (e.g., by the processor 104) to control the robotic arm 116 or components thereof (e.g., the arm end 230, the force transducer 234, the end effector 236) and/or the surgical tool 136. The forces detected and measured by the force transducer 234 may be processed by the computing device 102 to determine the resulting force on the surgical tool 136 and/or the end effector 236 and, when the resulting force is greater than a threshold value, the computing device 102 may take generate an alert. The alert may indicate that the robotic arm 116 or components thereof, the surgical tool 136, and/or patient anatomy (e.g., the vertebra 304) experienced a deflection. In some embodiments, the alert may be rendered to a display (e.g., the user interface 110) to inform the user (e.g., a surgeon, a member of the surgical staff, etc.) that the system 100 has detected a deflection. In some embodiments, one or more components of the system 100 may take one or more corrective actions based on the alert. For example, the processor 104 may cause the navigation system 118 to adjust the tracked pose of the robotic arm 116 or components thereof and/or the surgical tool 136. In other words, the processor 104 may determine the new positions of the arm end 230, the end effector 236, and/or the surgical tool 136 based on the deflection, and update the navigation system 118 with the new position(s). Based on the deflection and the detection of the alert, the navigation system 118 may automatically disable the operation of the surgical tool 136.

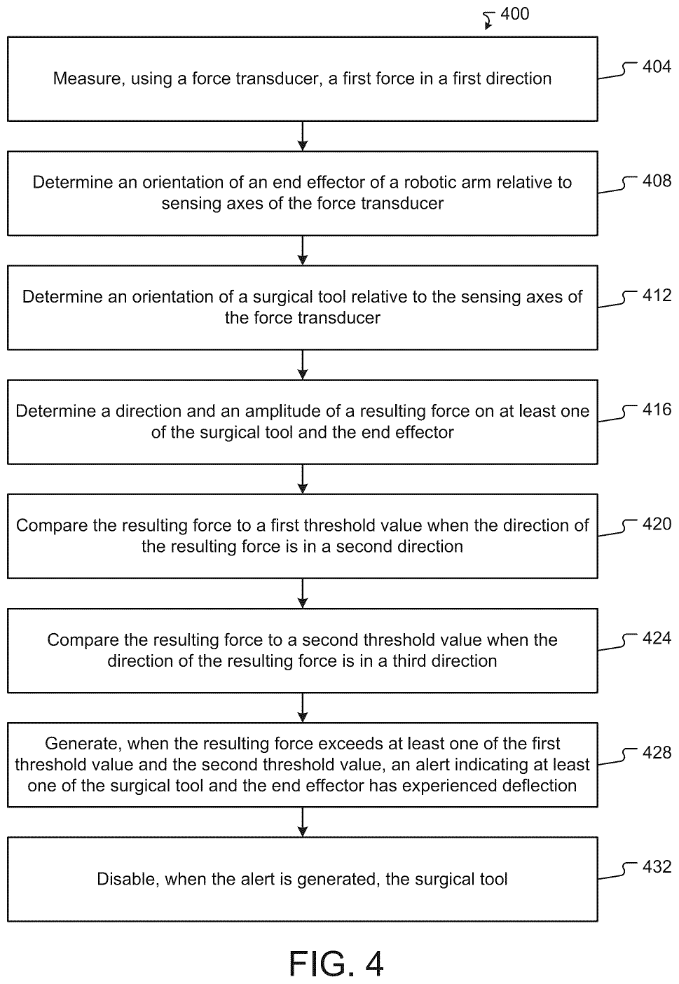

FIG. 4 depicts a method 400 that may be used, for example, to determine whether forces on a robotic arm and/or surgical tool result in one or more arm deflections.

The method 400 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 400. The at least one processor may perform the method 400 by executing elements stored in a memory such as the memory 106. The elements stored in memory and executed by the processor may cause the processor to execute one or more steps of a function as shown in method 400. One or more portions of a method 400 may be performed by the processor executing any of the contents of memory, such as an image processing 120, a segmentation 122, a transformation 124, and/or a registration 128.

The method 400 comprises measuring, using a force transducer, a first force in a first direction (step 404). The force transducer may be similar to or the same as the force transducer 234. The first force may be similar to or the same as the force 336. The force 336 may be measured along one or more sensing axes 340 of the force transducer 234.

The method 400 also comprises determining an orientation of an end effector of a robotic arm relative to sensing axes of the force transducer (step 408). The robotic arm may be similar to or the same as the robotic arm 116, the end effector may be similar to or the same as the end effector 236, and the sensing axes may be similar to or the same as the sensing axes 340. The orientation of the end effector 236 relative to the force transducer 234 may be determined based on measurements of one or more pose sensors (e.g., sensors that measure changes in position and/or orientation) disposed on or proximate the end effector 236, based on the navigation and/or tracking of the end effector 236 by the navigation system 118, combinations thereof, and the like. Once the orientation of the end effector 236 relative to the force transducer 234 is determined, the orientation of the end effector 236 relative to the sensing axes 340 of the force transducer 234 may be determined based on the known orientation of the sensing axes 340 of the force transducer 234 relative to the force transducer 234.

The method 400 also comprises determining an orientation of a surgical tool relative to the sensing axes of the force transducer (step 412). The surgical tool may be similar to or the same as the surgical tool 136. In some embodiments, the step 412 may be similar to the step 408. In other words, the orientation of the surgical tool 136 relative to the force transducer 234 may be determined based on measurements of one or more pose sensors (e.g., sensors that measure changes in position and/or orientation) disposed on or proximate the surgical tool 136, based on the navigation and/or tracking of the surgical tool 136 by the navigation system 118, combinations thereof, and the like. After the orientation of the surgical tool 136 relative to the force transducer 234 is determined, the orientation of the surgical tool 136 relative to the sensing axes 340 of the force transducer 234 may be determined based on the known orientation of the sensing axes 340 of the force transducer 234 relative to the force transducer 234.

The method 400 also comprises determining a direction and an amplitude of a resulting force on at least one of the surgical tool and the end effector (step 416). Since the orientation of the sensing axes 340 relative to the surgical tool 136 and/or the end effector 236 has been determined, the measured forces on the sensing axes 340 may be used to determine the resulting force amplitude and direction on the surgical tool 136 and/or the end effector 236. In some embodiments, the resulting force may be determined based on the mechanical coupling of the surgical tool 136 to the end effector 236, the mechanical coupling of the end effector 236 to the force transducer 234, combinations thereof, and the like.

The method 400 also comprises comparing the resulting force to a first threshold value when the direction of the resulting force is in a second direction (step 420). The first threshold value may represent the tolerance of the system, such that forces above the first threshold value are treated by the system as sufficient to generate a deflection in the surgical tool 136 and/or the end effector 236. The second direction may be, for example, a lateral direction, an axial direction, combinations thereof, and the like. The resulting force may be compared to the first threshold value to determine whether the resulting force is sufficient to generate a deflection.

The method 400 also comprises comparing the resulting force to a second threshold value when the direction of the resulting force is in a third direction (step 424). The second threshold value may represent the tolerance of the system, such that forces above the second threshold value are treated by as sufficient to generate a deflection in the surgical tool 136 and/or the end effector 236. The third direction may be, for example, a lateral direction, an axial direction, combinations thereof, and the like. The resulting force may be compared to the first threshold value to determine whether the resulting force is sufficient to generate a deflection. In some embodiments, the different threshold values may be used due to the different directions of the forces and the differing propensities of the forces to cause a deflection due to the mechanical nature of the surgical tool 136 and/or the end effector 236. For example, the second direction may be an axial force on the surgical tool 136 and/or the end effector 236, with such a direction having a greater inertial moment (e.g., a greater force is required to result in the same degree of movement or deflection) than in other directions. In contrast, the third direction may be a lateral force on the surgical tool 136 and/or the end effector 236, such that the magnitude of the force required to cause a deflection is lower than when the force occurs in the axial direction. As a result, the second threshold value may be lower than the first threshold value to account for the lower inertial moment of the surgical tool 136 and/or the end effector 236 in the lateral direction.

The method 400 also comprises generating, when the resulting force exceeds at least one of the first threshold value and the second threshold value, an alert indicating at least one of the surgical tool and the end effector has experienced deflection (step 428). When the resulting force exceeds at least one of the first threshold value and the second threshold value, the system 100 may treat the resulting force for sufficient to cause a deflection in the surgical tool 136, the end effector 236, and/or patient anatomy. In some embodiments, the resulting force may be treated as causing a deflection regardless of whether or not a deflection actually occurred. The alert may indicate that the resulting force has caused the deflection, and may be rendered in some cases to a display (e.g., user interface 110) to indicate that a deflection as occurred.

The method 400 also comprises disabling, when the alert is generated, the surgical tool (step 432). The generation of the alert may indicate that the surgical tool 136 and/or the end effector 236 has been deflected. The processor 104 and/or the navigation system 118 may automatically disable the surgical tool 136 when the alert is generated. Additionally or alternatively, the processor 104 and/or the navigation system 118 may cause the robotic arm 116 to move such that the operative portion 332 of the surgical tool 136 no longer interacts with the vertebra 304. The movement of the robotic arm 116 and/or the disabling of the surgical tool 136 may occur to prevent or mitigate the probability of patient harm, such as when the deflection to the operative portion 332 relative to the vertebra 304 occurs.

The present disclosure encompasses embodiments of the method 400 that comprise more or fewer steps than those described above, and/or one or more steps that are different than the steps described above.

As noted above, the present disclosure encompasses methods with fewer than all of the steps identified in FIG. 4 (and the corresponding description of the method 400), as well as methods that include additional steps beyond those identified in FIG. 4 (and the corresponding description of the method 400). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

The techniques of this disclosure may also be described in the following examples.

Example 1: A system (100), comprising:
a robotic arm (116) that includes a distal end interface (230);
a force transducer (234) couplable to the distal end interface (230) and that measures forces exerted on the force transducer (234) in up to six degrees of freedom; and
an end effector (236) configured to be in force-transmitting contact with the force transducer (234).

Example 2: The system according to example 1, wherein the end effector (236) is connectable to a surgical tool (136).

Example 3: The system according to examples 1 or 2, further comprising:
a processor (104); and
a memory (106) coupled to the processor (104) and storing data thereon that, when executed by the processor (104), enable the processor (104) to:
determine an orientation of the end effector (236) relative to sensing axes (340) of the force transducer (234); and
determine an orientation of a surgical tool (136) relative to the sensing axes (340) of the force transducer (234).

Example 4: The system according to example 3, wherein the data further enable the processor (104) to:
determine, when the force transducer (234) measures a first force in a first direction and based on the orientation of the surgical tool (136) relative to the sensing axes (340), a direction and an amplitude of a resulting force on the surgical tool (136).

Example 5: The system according to example 4, wherein the data further enable the processor (104) to:
compare the resulting force to a first threshold value when the direction of the resulting force on the surgical tool (136) is in a second direction;
compare the resulting force to a second threshold value when the direction of the resulting force on the surgical tool (136) is in a third direction; and
generate, when the resulting force exceeds at least one of the first threshold value and the second threshold value, an alert indicating the surgical tool (136) has experienced deflection.

Example 6: The system according to example 5, wherein the data further enable the processor (104) to:
automatically disable, when the alert is generated, the surgical tool (136).

Example 7: The system according to any of examples 3 to 6, wherein the data further enable the processor (104) to:
determine, when the force transducer (234) measures a first force in a first direction and based on the orientation of the end effector (236) relative to the sensing axes (340), a direction and an amplitude of a resulting force on the end effector (236).

Example 8: The system according to example 7, wherein the data further enable the processor (104) to:
compare the resulting force to a first threshold value when the direction of the resulting force on the end effector (236) is in a second direction;
compare the resulting force to a second threshold value when the direction of the resulting force on the end effector (236) is in a third direction; and
generate, when the resulting force exceeds at least one of the first threshold value and the second threshold value, an alert indicating the end effector (236) has experienced deflection.

Example 9: An apparatus, comprising:
a robotic arm (116) with a first end;
a force transducer (234) with a first end in force-transmitting contact with the first end of the robotic arm (116), the force transducer (234) measuring forces exerted on the force transducer (234) in up to six degrees of freedom; and
an end effector (236) with a first end in force-transmitting contact with a second end of the force transducer (234), wherein the end effector (236) includes a second end that is couplable to a surgical instrument (136).

Example 10: The apparatus according to example 9, further comprising:
a processor (104); and
a memory (106) coupled to the processor (104) and storing data thereon that, when executed by the processor (104), enable the processor (104) to:
determine an orientation of the end effector (236) relative to sensing axes (340) of the force transducer (234); and
determine an orientation of a surgical instrument (136) relative to the sensing axes (340) of the force transducer (234).

Example 11: The apparatus according to example 10, wherein the data further enable the processor (104) to:
determine, when the force transducer (234) measures a first force in a first direction and based on the orientation of the surgical instrument (136) relative to the sensing axes (340), a direction and an amplitude of a resulting force on the surgical instrument (136).

Example 12: The apparatus according to example 11, wherein the data further enable the processor (104) to:
compare the resulting force to a first threshold value when the direction of the resulting force on the surgical instrument (136) is in a second direction;
compare the resulting force to a second threshold value when the direction of the resulting force on the surgical instrument (136) is in a third direction; and
generate, when the resulting force exceeds at least one of the first threshold value and the second threshold value, an alert indicating the surgical instrument (136) has experienced deflection.

Example 13: The apparatus according to example 12, wherein the data further enable the processor (104) to:
automatically disable, when the alert is generated, the surgical instrument (136).

Example 14: The apparatus according to any of examples 10 to 13, wherein the data further enable the processor (104) to:
determine, when the force transducer (234) measures a first force in a first direction and based on the orientation of the end effector (236) relative to the sensing axes (340), a direction and an amplitude of a resulting force on the end effector (236).

Example 15: The apparatus according to example 14, wherein the data further enable the processor (104) to:
compare the resulting force to a first threshold value when the direction of the resulting force on the end effector (236) is in a second direction;
compare the resulting force to a second threshold value when the direction of the resulting force on the end effector (236) is in a third direction; and
generate, when the resulting force exceeds at least one of the first threshold value and the second threshold value, an alert indicating the end effector (236) has experienced deflection.

Various examples of the disclosure have been described. These and other examples are within the scope of the following claims.

What is claimed is:

1. A system, comprising:
a robotic arm that includes a distal end interface;
a force transducer couplable to the distal end interface and that measures forces exerted on the force transducer in up to six degrees of freedom; and
an end effector configured to be in force-transmitting contact with the force transducer.

2. The system of claim 1, wherein the end effector is connectable to a surgical tool.

3. The system of claim 1, further comprising:
a processor; and
a memory coupled to the processor and storing data thereon that, when executed by the processor, enable the processor to:
determine an orientation of the end effector relative to sensing axes of the force transducer; and
determine an orientation of a surgical tool relative to the sensing axes of the force transducer.

4. The system of claim 3, wherein the data further enable the processor to:
determine, when the force transducer measures a first force in a first direction and based on the orientation of the surgical tool relative to the sensing axes, a direction and an amplitude of a resulting force on the surgical tool.

5. The system of claim 4, wherein the data further enable the processor to:
compare the resulting force to a first threshold value when the direction of the resulting force on the surgical tool is in a second direction;
compare the resulting force to a second threshold value when the direction of the resulting force on the surgical tool is in a third direction; and
generate, when the resulting force exceeds at least one of the first threshold value and the second threshold value, an alert indicating the surgical tool has experienced deflection.

6. The system of claim 5, wherein the data further enable the processor to:
automatically disable, when the alert is generated, the surgical tool.

7. The system of claim 3, wherein the data further enable the processor to:
determine, when the force transducer measures a first force in a first direction and based on the orientation of the end effector relative to the sensing axes, a direction and an amplitude of a resulting force on the end effector.

8. The system of claim 7, wherein the data further enable the processor to:
compare the resulting force to a first threshold value when the direction of the resulting force on the end effector is in a second direction;
compare the resulting force to a second threshold value when the direction of the resulting force on the end effector is in a third direction; and
generate, when the resulting force exceeds at least one of the first threshold value and the second threshold value, an alert indicating the end effector has experienced deflection.

9. An apparatus, comprising:
a robotic arm with a first end;
a force transducer with a first end in force-transmitting contact with the first end of the robotic arm, the force transducer measuring forces exerted on the force transducer in up to six degrees of freedom; and
an end effector with a first end in force-transmitting contact with a second end of the force transducer, wherein the end effector includes a second end that is couplable to a surgical instrument.

10. The apparatus of claim 9, further comprising:
a processor; and
a memory coupled to the processor and storing data thereon that, when executed by the processor, enable the processor to:
determine an orientation of the end effector relative to sensing axes of the force transducer; and
determine an orientation of the surgical instrument relative to the sensing axes of the force transducer.

11. The apparatus of claim 10, wherein the data further enable the processor to:
determine, when the force transducer measures a first force in a first direction and based on the orientation of the surgical instrument relative to the sensing axes, a direction and an amplitude of a resulting force on the surgical instrument.

12. The apparatus of claim 11, wherein the data further enable the processor to:
compare the resulting force to a first threshold value when the direction of the resulting force on the surgical instrument is in a second direction;
compare the resulting force to a second threshold value when the direction of the resulting force on the surgical instrument is in a third direction; and
generate, when the resulting force exceeds at least one of the first threshold value and the second threshold value, an alert indicating the surgical instrument has experienced deflection.

13. The apparatus of claim 12, wherein the data further enable the processor to:
automatically disable, when the alert is generated, the surgical instrument.

14. The apparatus of claim 10, wherein the data further enable the processor to:
determine, when the force transducer measures a first force in a first direction and based on the orientation of the end effector relative to the sensing axes, a direction and an amplitude of a resulting force on the end effector.

15. The apparatus of claim 14, wherein the data further enable the processor to:
compare the resulting force to a first threshold value when the direction of the resulting force on the end effector is in a second direction;
compare the resulting force to a second threshold value when the direction of the resulting force on the end effector is in a third direction; and
generate, when the resulting force exceeds at least one of the first threshold value and the second threshold value, an alert indicating the end effector has experienced deflection.

16. A surgical system, comprising:
a robotic arm that includes a distal end interface;
a force transducer couplable to the distal end interface and that measures forces exerted on the force transducer in up to six degrees of freedom;
an end effector configured to be in force-transmitting contact with the force transducer and couplable to a surgical tool;
a processor; and
memory coupled to the processor and storing data thereon that enable the processor to:
determine an orientation of the surgical tool relative to sensing axes of the force transducer; and
determine an orientation of the end effector relative to the sensing axes of the force transducer.

17. The surgical system of claim 16, wherein the data further enable the processor to:
determine, when the force transducer measures a first force in a first direction and based on at least one of the orientation of the surgical tool relative to the sensing axes and the orientation of the end effector relative to the sensing axes, a direction and an amplitude of a resulting force on at least one of the surgical tool and the end effector.

18. The surgical system of claim 17, wherein the data further enable the processor to:
compare the resulting force to a first threshold value when the direction of the resulting force is in a second direction;
compare the resulting force to a second threshold value when the direction of the resulting force is in a third direction; and
generate, when the resulting force exceeds at least one of the first threshold value and the second threshold value, an alert indicating at least one of the surgical tool and the end effector has experienced deflection.

19. The surgical system of claim 18, wherein the data further enable the processor to:
automatically disable, when the alert is generated, the surgical tool.

20. The surgical system of claim 18, wherein the second direction is along an axial direction of the surgical tool, and wherein the third direction is lateral to the surgical tool.
